(19) United States
(12) Patent Application Publication
(10) Pub. No.: US 2008/0160102 A1
(43) Pub. Date: Jul. 3, 2008

(54) Compositions For Disfecing Inanimate Surfaces

(76) Inventors: Igor Anatolievich Pomytkin, Moscow (RU); Sergey Pavlovich Soloviev, Moscow (RU)

Correspondence Address: NOTARO AND MICHALOS, 100 DUTCH HILL ROAD, SUITE 110, ORANGEBURG, NY, 10962-2100, US

(21) Appl. No.: 11/817,908

(22) PCT Filed: Mar. 11, 2005
(86) PCT No.: PCT/RU05/00106

§ 371 Date: Nov. 15, 2007

(51) Int. Cl. A61K 33/00; A61K 33/40

(52) U.S. Cl. 424/616; 514/694; 514/724; 514/643; 514/636; 424/661; 424/600

(57) ABSTRACT

This invention relates to compositions for disinfecting an inanimate surface comprising an effective disinfecting amount of a disinfectant and water, which water comprises from about 99.760 to about 99.999% of light isotopologue ¹H₂ ¹⁶0 and up to 100% of residual isotopologues ¹H₂¹⁷0, ¹H₂¹⁸0, ¹H²H¹⁶0, ¹H²H¹⁷0, ¹H²H¹⁸0, ²H₂¹⁶0, ²H₂¹⁷0, and ²H₂¹⁸0. The disinfectant is selected from the group consisting of aldehydes, alcohols, phenol compounds, quaternary ammonium compounds, chlorhexidine, halogen compounds, peroxides and hydroperoxides. Further, the invention relates to method of disinfecting an inanimate surface, which comprises a step of contacting said surface with the composition of the present invention.

### TECHNICAL FIELD

The present invention relates to compositions for disinfecting inanimate surfaces.

### BACKGROUND OF THE INVENTION

Disinfection is a generally recognized process by which viable pathogens, e.g. bacteria, are reduced to a level unlikely to produce disease in healthy people, plants, or animals.

It is known from the art that disinfecting compositions have a variety of multi-industrial, hospital, and consumer uses. So, disinfecting compositions are used to disinfect and clean various inanimate surfaces including hard surfaces like wails, tiles, table tops, glass, bathroom surfaces, kitchen surfaces, dishes as well as fabrics, clothes, carpets and the like; ventilation means surfaces; air duct surfaces; fruit and vegetable surfaces; hospital rooms and surfaces; surfaces of heat-sensitive medicinal equipment like endoscopes; surfaces of contact lenses; and etc. Rutala W A et al., Am J Infect Control. 2004 32(4):226-31. Rutala W A et al, Infect Control Hosp Epidemiol. 1999 20(1):69-76. Cefai C et al., J Hosp Infect. 1990 15(2):177-82. Rutala W A et al., J Hosp Infect. 2001 48 Suppl A:S64-8. Beuchat, L. R. 1998. Food Safety Unit, WHO. Report WHO/FSF/FOS/98.2.

Chemical disinfectants are used for the disinfection of surfaces of inanimate object that cannot be heated or autoclaved. The most widely used chemical disinfectants are oxidative agents like hydrogen peroxide, organic and inorganic peroxides, ozone, hypochlorite, chlorine, chlorine dioxide, chloramines, bromine and derivatives thereof, iodine and derivatives thereof Another class of disinfectant is organic compounds like formaldehyde, glutaraldehyde, quaternary ammonium compounds, phenol compounds, alcohols, and etc. Rutala W A. APIC Guideline for Selection and Use of Disinfectants. Am J Infect Control 1996 24(4):313-42.

It is known from the art that disinfectants may induce adverse effects such as skin irritation, allergies, and asthma in humans and/or animals. Daschner F et al., Am J Infect Control. 2004 32(4):224-5.

Thus, there is the need to reduce the adverse effects of disinfecting compositions.

Generally, disinfecting compositions are formulated in liquid form with water as the most widely used solvent.

As known from the art, natural water is a mixture of nine water isotopologues (¹H₂¹⁶O, ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O) formed by stable isotopes of hydrogen (¹H and ²H) and oxygen (¹⁶O, ¹⁷O, ¹⁸O), wherein the level of light water isotopologue ¹H₂¹⁶O is about 99.7317% (Vienna Standard Mean Ocean Water, VSMOW), and wherein total level of all eight heavy isotopologues comprising at least one heavy isotopes ²H, ¹⁷O, and ¹⁸O is about 0.2683% (e.g. 0.199983% ¹H₂¹⁸O, 0.0372% ¹H₂¹⁷O, 0.031069% ¹H²H ¹⁶O, 0.0000623% ¹H²H ¹⁸O, and 0.0000116% ¹H²H ¹⁷O). Rothman et al., J. Quant. Spectrosc. Radiat. Transfer, 1998, 60, 665. Rothman et al., J. Quant. Spectrosc. Radiat. Transfer, 2003, 82, p. 9. The abundance of water isotopologues in natural water slightly varies on Earth district and climatic conditions and is expressed typically as the deviation, δ, relative to the international VSMOW standard. The natural water enriched maximally by major light water isotopologue ¹H₂¹⁶O was founded in Antarctica (Standard Light Antarctic Precipitation, SLAP), wherein said δ-values of residual heavy isotopes are δ²H −415.5%₀, δ¹⁷O −28.1%₀, and δ¹⁸O −53.9%₀ that corresponds to the 99.757% level of light water isotopologue ¹H₂¹⁶O. R. van Trigt, Laser Spectrometry for Stable Isotope Analysis of Water Biomedical and Paleoclimatological Applications, 2002, Groningen: University Library Groningen, p. 50.

Thus, typical abundance of light water isotopologue ¹H₂¹⁶O in natural water is about 99.73% and water with the abundance of light water isotopologue ¹H₂¹⁶O more than 99.757% is not found in nature. Accordingly, all known from the art disinfecting compositions comprise water with level of light water isotopologue ¹H₂¹⁶O about 99.73% and never more than 99.757%. Accordingly, disinfecting compositions, which comprise water with level of light water isotopologue ¹H₂¹⁶O from 99.760 to 99.999%, are unlnown from the art.

Water with content of light water isotopologue ¹H₂¹⁶O equal or more than 99.76% can be prepared by industrial methods providing depletion of natural water of heavy isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O.

We found that water enriched by light water isotopologue ¹H₂¹⁶O to the level unknown in nature (from 99.760 to 99.999%) potentiates the effect of a disinfectant. Because of potentiating the effect of the disinfectant, the use of the water enriched by light water isotopologue ¹H₂¹⁶O provides particularly advantageous compositions for achieving the disinfecting effect with less than usually effective amounts of the disinfectant. Therefore, it is possible to minimize potential adverse effects, which may be associated with larger amounts of disinfectant and still achieve the disinfecting effect.

It is an object of the present invention to provide a composition for disinfecting an inanimate surface comprising an effective disinfecting amount of a disinfectant and water, which water comprises from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O.

### BRIEF DESCRIPTION OF THE DRAWINGS

FIG. 1 is a schematic side view of an apparatus for the manufacturing the water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O.

### DISCLOSURE OF INVENTION

The present invention provides a composition for disinfecting an inanimate surface comprising an effective disinfecting amount of a disinfectant and water, which water comprises from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O.

As used herein, the term “isotopologue” is in accordance with IUPAC Compendium of Chemical Terminology 2nd Edition (1997) and refers to a molecular entity that differs only in isotopic composition (number of isotopic substitutions), e.g. ¹H₂¹⁶O, ¹H²H¹⁶O, and ¹H₂¹⁸O.

As used herein, the term “disinfectant” means an agent that destroys or inhibits pathogens capable of replication and capable of causing diseases in humans, animals, or plants. Examples of such pathogens include, but are not limited to, bacteria, spores, fungi, cells, viruses, DNA, biologically active substances, and prions. Suitable disinfectants are all those known by those skilled in the art for the purpose of disinfecting. A generally recognized compendium of such disinfectants is APIC Guideline for Selection and Use of Disinfectants. Rutala W A. Am J Infect Control 1996 24(4):313-42.

In the composition of the invention, the disinfectant is selected from the group consisting of aldehydes, alcohols, phenol compounds, quaternary ammonium compounds, chlorhexidine, halogen compounds, peroxides and hydroperoxides.

Such aldehydes include, but are not limited to, formaldehyde, glutaraldehyde, and glyoxal.

Such alcohols include, but are not limited to, ethanol, propanol, and isopropanol.

Such phenol compounds include, but are not limited to, m-cresol, p-chloro-m-cresol, and p-chloro-m-xylenol.

Such quaternary ammonium compounds include, but are not limited to, benzalkonium chloride, quaternary ammonium compounds containing alkyl or substituted alkyl groups, alkyl amide and carboxylic acid groups, ether groups, unsaturated alkyl groups, and cyclic quaternary ammonium compounds like as alkylpyridinium chlorides and/or sulphates, alkylisoquinolyl chlorides and/or bromides.

Such halogen compounds include, but are not limited to, chlorine, chlorine dioxide, sodium hypochlorite, chloramines, and iodophor.

Such peroxides and hydroperoxides include, but are not limited to, hydrogen peroxide or salts thereof; organic peroxides like as peracetic acid, peroxyacids, dialkylperoxides, diacylperoxides; and inorganic peroxides like as perborates, percarbonates, persilicates, persulphates, and potassium peroxomono-sulphate.

As used herein, the term “effective disinfecting amount” means an amount sufficient to allow the disinfectant to perform its action, i.e. to reduce the number of micro-organisms existing on a given surface. Depending on the disinfectant used the amount used may be different. Typically, the effective disinfecting amount is from 0.001% to 90% by total weight of the composition, preferably from 0.05% to 90%.

The effective amounts of the aldehydes (e.g. formaldehyde, glutaraldehyde, and glyoxal) are generally used in the composition at levels from 0.2 to 8% by total weight of the composition.

The effective amounts of the alcohols (e.g. ethanol, propanol, and isopropanol) are generally used in the composition at levels from 70 to 90% by total weight of the composition.

The effective amounts of the phenol compounds (e.g. phenol, m-cresol, p-chloro-m-cresol, and p-chloro-m-xylenol) are generally used in the composition at levels from 0.1 to 5% by total weight of the composition.

The effective amounts of the quaternary ammonium compounds (e.g. benzalkonium chloride) are generally used in the composition at levels from 0.1 to 5% by total weight of the composition.

The effective amounts of chlorhexidine are generally used in the composition at levels from 0.05 to 0.5% by total weight of the composition.

The effective amounts of the halogen compounds (e.g. chlorine, chlorine dioxide, sodium hypochlorite, chloramines, and iodophor) are generally used in the composition at levels from 0.001 to 5% by total weight of the composition.

The effective amounts of the peroxides and hydroperoxides (e.g. H₂O₂, peracetic acid, and potassium peroxomono-sulphate) are generally used in the composition at levels from 0.02 to 3% by total weight of the composition.

Herein and after, term “light water” refers to water comprising from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O.

In practicing the invention, light water is prepared from the natural water comprising from about 99.732 to 99.757% of light isotopologue ¹H₂¹⁶O by methods known from the art. Preferably, the light water is prepared by distillation of the natural water.

The amount of light water in the composition to be employed according to this invention may be varied from 10 to 99.99% depending on the disinfectant used.

The amount of light water is generally used at levels from 92 to 99.8% by total weight of the composition comprising aldehydes (e.g. formaldehyde, glutaraldehyde, and glyoxal) as disinfectants.

The amount of light water is generally used at levels from 10 to 30% by total weight of the composition comprising alcohols (e.g. ethanol, propanol, and isopropanol) as disinfectants.

The amount of light water is generally used at levels from 95 to 99.9% by total weight of the composition comprising phenol compounds (e.g. phenol, m-cresol, p-chloro-m-cresol, and p-chloro-m-xylenol) as disinfectants.

The amount of light water is generally used at levels from 95 to 99.9% by total weight of the composition comprising quaternary ammonium compounds (e.g. benzalkonium chloride) as disinfectants.

The amount of light water is generally used at levels from 99.5 to 99.95% by total weight of the composition comprising chlorhexidine as disinfectants.

The amount of light water is generally used at levels from 95 to 99.99% by total weight of the composition comprising halogen compounds (e.g. chlorine, chlorine dioxide, sodium hypochlorite, chloramines, and iodophor) as disinfectants.

The amount of light water is generally used at levels from 97 to 99.98% by total weight of the composition comprising peroxides or hydroperoxides (e.g. H₂O₂, peracetic acid, and potassium peroxomono-sulphate) as disinfectants.

The composition of the invention further comprises a surfactant or mixtures thereof. Surfactants contribute to the cleaning performance of the disinfecting compositions of the present invention. Suitable surfactants to be used herein may be any those surfactant known to those skilled in the art. A generally recognized compendium of such surfactants is “Surface Active Agents and Detergents”, Vol. 1 and 2, by Schwartz, Perry and Berch.

Suitable surfactant is selected from the group consisting of anionic, nonionic, cationic, amphoteric and/or zwitterionic surfactants.

Suitable anionic surfactants include, but are not limited to, organic sulfates, sulfonates, and carboxylates. Preferred anionic surfactants for use in the compositions herein are salts or acids of the formula ROSO₃M wherein R is preferably a C₆-C₂₄ alkyl or hydroxyalkyl, and M is H or a cation, e.g. alkali metal cation (e.g. sodium, potassium, lithium), or ammonium or substituted ammonium (e.g., methyl-, dimethyl-, and trimethyl ammonium cations and quaternary ammonium cations, such as tetramethyl-ammonium and dimethyl piperdinium cations and quaternary ammonium cations derived from alkylamines such as ethylamine, diethylamine, triethylamine, and mixtures thereof); alkyl benzene sulfonates; allyl alkoxylated sulfates; paraffin sulfonates; alkyl-carboxylates and mixtures thereof.

Suitable cationic surfactants include, but are not limited to, alkyl amines, monalkyl quaternary amines, di-alkyl quaternary amines, tri-alkyl quaternary amines, imidazolinium quaternary amines, and combinations thereof. Preferred cationic surfactant to be used according to the present invention is cetyltrimethylammonium bromide.

Suitable nonionic surfactants include, but are not limited to, amine oxides having the formula R₁R₂R₃NO wherein each of R₁, R₂ and R₃ is independently a saturated substituted or unsubstituted, linear or branched hydrocarbon chain of from 1 to 30 carbon atoms. Preferred amine oxide surfactants to be used according to the present invention are amine oxides of the formula R₁R₂R₃NO wherein R₁ is an hydrocarbon chain comprising from 1 to 30 carbon atoms, preferably from 8 to 12, and wherein R₂ and R₃ are independently substituted or unsubstituted, linear or branched hydrocarbon chains comprising from 1 to 4 carbon atoms, preferably are methyl groups. R₁ may be a saturated substituted or unsubstituted, linear or branched hydrocarbon chain. Suitable amine oxides for use herein are for instance natural blend C₈-C₁₀ amine oxides as well as C₁₂-C₁₆ amine oxides commercially available from Hoechst.

Suitable amphoteric and/or zwitterionic surfactants include, but are not limited to, betaine and sulphobetaine surfactants, derivatives thereof or mixtures thereof.

The compositions preferably have a total surfactant level of from 0.5% to 75% by weight, more preferably from 1% to 50% by weight, most preferably from 5% to 30% by weight of total composition.

The composition further comprises an optional component selected from the group consisting of chelating agents, pH buffering agents, stabilizers, perfumes, enzymes, pigments, dyes, and mixtures thereof.

Suitable chelating agents may be any of those known to those skilled in the art such as the ones selected from the group comprising phosphonate chelating agents, amino carboxylate chelating agents or other carboxylate chelating agents, or polyfunctionally-substituted aromatic chelating agents or mixtures thereof. Preferred chelating agent to be used according to the present invention is ethylenediaminetetraacetates (EDTA). Typically, the compositions according to the present invention comprise up to 5% by weight of the total composition of a chelating agent, or mixtures thereof, preferably from 0.002% to 3% by weight.

Suitable pH buffering agents may be any of those known to those skilled in the art such as the ones selected from the group comprising include organic acids and mixtures thereof, phosphate buffer, bicarbonate buffer. Suitable organic acids for use herein include monocarboxylic acids, dicarboxylic acids and tricarboxylic acids or mixtures thereof. Preferred organic acids for use herein include acetic acid, citric acid, malonic acid, maleic acid, malic acid, lactic acid, glutaric acid, glutamic acid, aspartic acid, methyl succinic acid, succinic acid or mixtures thereof. The compositions are formulated in the pH range 2 to 12, preferably from 2 to 9. Typically, the compositions according to the present invention may comprise up to 15% by weight of the total composition of a pH buffer, or mixtures thereof, preferably from 0.1% to 3%.

The compositions of the invention are prepared by known in the art procedures. In making the compositions, the disinfectant will usually be mixed with the light water. Further, other components such as surfactants, chelating agents, pH buffering agents, stabilizers, perfumes, enzymes, pigments, dyes, and mixtures thereof may be added to the composition.

The compositions herein may be packaged in a variety of suitable packaging known to those skilled in the art. Such packagings include, but are not limited to, manually operated spray dispensing containers and solution containers. Preferred spray dispenser is a pump spray dispenser.

The disinfecting properties of the composition of the present invention may be measured by the bactericidal activity of said composition. A test method suitable to evaluate the bactericidal activity of a composition on infected surfaces is described in European Standards (e.g. prEN 1040, CEN/TC 216 N 78, November 1995).

The present invention further provides a method of disinfecting an inanimate surface, which comprises a step of contacting said surface with the composition comprising an effective disinfecting amount of a disinfectant and water, which water comprises from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂₁₆O, ²H₂¹⁷O, and ²H₂¹⁸O.

The inanimate surface to disinfect with the compositions herein may be any of known to those skilled in the art such as typically found in houses like kitchens, bathrooms, e.g., tiles, walls, floors, chrome, glass, smooth vinyl, any plastic, plastified wood, table top, sinks, cooker tops, dishes, sanitary fittings such as sinks, showers, shower curtains, wash basins, WCs and the like; refrigerators, freezers, washing machines, automatic dryers, ovens, microwave ovens, dishwashers, ventilators and so on; hospital rooms and surfaces; dental and medical equipment such as instruments for surgery, endoscopes and the like; contact lenses; food products such as meats, poultry, fish, fruits, and vegetables; and other surfaces which require hygienic and sanitary conditions to be properly handled and/or used.

In practicing the method of the invention, disinfecting composition, as described herein, needs to be contacted with the inanimate object to be disinfected. The composition in the liquid form may be applied to the surface to be disinfected in their neat form or in their diluted form typically at a dilution level up to 100 times their weight of light water, preferably into 60 to 10 times their weight of light water. Depending on the disinfectant used the time of contacting may be different. Typically, the time of contacting is 1 min to 6 hours. Preferably, the time of contacting is about 10 min to 6 hours for aldehydes as disinfectants, from 1 to 30 min for alcohols as disinfectants, from 1 min to 2 hours for peroxides as disinfectants, from 10 to 30 min for halogen compounds as disinfectants, from about 10 to 30 min for phenol compounds as disinfectants, and from 10 to 30 min for quaternary ammonium compounds.

The following examples are presented to demonstrate the invention. The examples are illustrative only and are not intended to limit the scope of the invention in any way.

### EXAMPLE 1

This example demonstrates the method for producing light water of the invention.

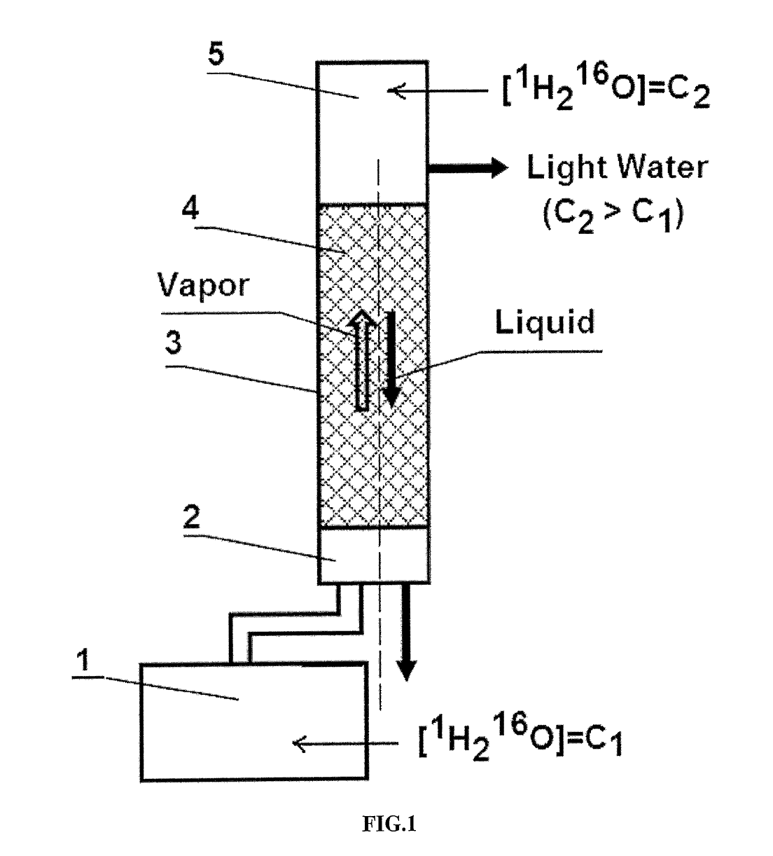

The light water comprising 99.99% of light isotopologue ¹H₂¹⁶O is prepared by distillation of natural water comprising 99.73% of light isotopologue ¹H₂¹⁶O with using the apparatus of FIG. 1 under temperature 60° C. and pressure 0.2 bars. The process of the distillation comprises evaporating natural water comprising 99.71% (C₁) of light isotopologue ¹H₂¹⁶O in boiling means 1 to produce water vapor; supplying the water vapor to the bottom 2 of distillation column 3; carrying out vapor-liquid contact between a descending liquid and an ascending vapor mainly on the surface of the contact device 4 (e.g. structured or random packing) within the distillation column, at which time the liquid and the vapor flow in mutually opposite directions over the surface of the contact device along a main flow direction which is along a direction of the column axis; condensing water vapor with concentration of light isotopologue ¹H₂¹⁶O 99.99% (C₂) on condenser 5 installed on upper bound of the distillation column 3; and collecting a part of condensate as condensed light water comprising 99.99% of light isotopologue ¹H₂¹⁶O (C₂>C₁) appropriate for the use in the compositions of the invention.

### EXAMPLE 2

This example shows a representative disinfecting composition comprising formaldehyde.

| | % wt. |
|---|---|
| Formaldehyde | 0.5 |
| Light water (99.99% of light isotopologue ¹H₂¹⁶O) | 99.5 |

The method for preparing the composition described in Example 2 is as follows: gaseous formaldehyde is introduced into light water up to level of 0.5%.

The method of disinfecting inanimate surface is as follows: the composition of the Example 2 contacts with the inanimate surface for 10 min.

### EXAMPLE 3

This example shows a representative disinfecting composition comprising ethyl alcohol.

| | % wt. |
|---|---|
| Ethyl alcohol | 70 |
| Light water (99.99% of light isotopologue ¹H₂¹⁶O) | 30 |

The method for preparing the composition described in Example 3 is as follows: ethyl alcohol and light water are mixed.

The method of disinfecting inanimate surface is as follows: the composition of the Example 3 contacts with the inanimate surface for 10 min.

### EXAMPLE 4

This example shows a representative disinfecting composition comprising m-cresol.

| | % wt. |
|---|---|
| m-Cresol | 1 |
| Light water (99.99% of light isotopologue ¹H₂¹⁶O) | 99 |

The method for preparing the composition described in Example 4 is as follows: m-cresol and light water are mixed.

The method of disinfecting inanimate surface is as follows: the composition of the Example 4 contacts with the inanimate surface for 30 min.

### EXAMPLE 5

This example shows a representative disinfecting composition comprising benzalkonium chloride.

| | % wt. |
|---|---|
| Benzalkonium chloride | 0.5 |
| Light water (99.99% of light isotopologue ¹H₂¹⁶O) | 99.5 |

The method for preparing the composition described in Example 5 is as follows: benzalkonium chloride and light water are mixed.

The method of disinfecting inanimate surface is as follows: the composition of the Example 5 contacts with the inanimate surface for 10 min.

### EXAMPLE 6

This example shows a representative disinfecting composition comprising chlorhexidine.

| | % wt. |
|---|---|
| Chlorhexidine | 0.05 |
| Light water (99.99% of light isotopologue ¹H₂¹⁶O) | 99.95 |

The method for preparing the composition described in Example 6 is as follows: chlorhexidine and light water are mixed.

The method of disinfecting inanimate surface is as follows: the composition of the Example 6 contacts with the inanimate surface for 30 min.

### EXAMPLE 7

This example shows a representative disinfecting composition comprising sodium hypochlorite.

| | % wt. |
|---|---|
| Sodium hypochlorite | 0.5 |
| Sodium dodecylsulfate (surfactant) | 1 |
| Light water (99.99% of light isotopologue ¹H₂¹⁶O) | 98.5 |

The method for preparing the composition described in Example 7 is as follows: sodium hypochlorite, sodium dodecylsulfate and light water are mixed.

The method of disinfecting inanimate surface is as follows: the composition of the Example 7 contacts with the inanimate surface for 30 min.

### EXAMPLE 8

This example shows a representative disinfecting composition comprising iodophor.

| | content |
|---|---|
| Iodophor | 25 ppm |
| Light water (99.99% of light isotopologue ¹H₂¹⁶O) | up to |
| | 100% |

The method for preparing the composition described in Example 8 is as follows: iodophor and light water are mixed.

The method of disinfecting inanimate surface is as follows: the composition of the Example 8 contacts with the inanimate surface for 10 min.

### EXAMPLE 9

This example shows a representative disinfecting composition comprising hydrogen peroxide.

| | % wt. |
|---|---|
| Hydrogen peroxide | 0.5 |
| Light water (99.99% of light isotopologue ¹H₂¹⁶O) | 99.5 |

The method for preparing the composition described in Example 9 is as follows: hydrogen peroxide and light water are mixed.

The method of disinfecting inanimate surface is as follows: the composition of the Example 9 contacts with the inanimate surface for 10 min.

The compositions exemplified above are according to the present invention. They provide disinfection at less amount of disinfectant than made on water with level of light isotopologue <99.760%. Therefore, it is possible to minimize potential adverse effects, which may be associated with larger amounts of disinfectant and still achieve the disinfecting effect.

1. A composition for disinfecting an inanimate surface comprising an effective disinfecting amount of a disinfectant and water, which water comprises from about 99.760 to about 99.999% of light isotopologue ¹H₂¹⁶O and up to 100% of residual isotopologues ¹H₂¹⁷O, ¹H₂¹⁸O, ¹H²H¹⁶O, ¹H²H¹⁷O, ¹H²H¹⁸O, ²H₂¹⁶O, ²H₂¹⁷O, and ²H₂¹⁸O.

2. The composition of claim 1, wherein the disinfectant is selected from the group consisting of aldehydes, alcohols, phenol compounds, quaternary ammonium compounds, chlorhexidine, halogen compounds, peroxides and hydroperoxides.

3. The composition of claim 1, further comprising a surfactant.

4. The composition of claim 3, wherein the surfactant is surfactant is selected from the group consisting of anionic, nonionic, cationic, amphoteric and/or zwitterionic surfactants.

5. The composition of claim 1, wherein the effective disinfecting amount of the disinfectant is from 0.001% to 90% by total weight of the composition.

6. The composition of claim 1, wherein the amount of said water is from 10 to 99.99% by total weight of the composition.

7. A method of disinfecting an inanimate surface, which comprises a step of contacting said surface with the composition of claim 1.
